(19) United States
(12) Patent Application Publication
(10) Pub. No.: US 2010/0285119 A1
(43) Pub. Date: Nov. 11, 2010

(54) Multiparticulate Extended Release Pharmaceutical Composition Of Carbamazepine And Process For Manufacturing The Same

(75) Inventor: Murali Krishna Bhavarisetti, Nandigama (IN)

Correspondence Address: COHEN, PONTANI, LIEBERMAN & PAVANE LLP, 551 FIFTH AVENUE, SUITE 1210, NEW YORK, NY, 10176, US

(73) Assignee: Jubilant Organosys Ltd., Noida, Uttar Pradesh (IN)

(21) Appl. No.: 12/812,330

(22) PCT Filed: Jan. 12, 2009
(86) PCT No.: PCT/IN2009/000035

§ 371 Date: Jul. 9, 2010

(30) Foreign Application Priority Data
Jan. 11, 2008 (IN) 104/DEL/2008

(51) Int. Cl. A61K 9/52; A61K 9/22; A61K 31/55; A61K 9/26; A61P 25/08; A61P 25/18; A61P 25/00

(52) U.S. Cl. 424/457; 424/468; 514/217; 424/465; 424/469

(57) ABSTRACT

Disclosed herein is an extended release pharmaceutical composition comprising at least two populations of extended release minitablets having a core comprising carbamazepine or its pharmaceutical salts, solvates, hydrates, an extended release polymer(s), wherein the extended release polymer of each population of minitablets is unalike. The process for manufacturing said composition is also disclosed.

### FIELD OF THE INVENTION

In general, the invention relates to a field of extended release pharmaceutical compositions and in particular, to an oral multiple unit extended release pharmaceutical composition of carbamazepine or its pharmaceutically acceptable salts, solvates, hydrates etc. Also provided is process for manufacturing of multiple unit extended release pharmaceutical composition of carbamazepine.

### BACKGROUND OF THE INVENTION

Carbamazepine is an iminostilbene derivative, which is used particularly as an antiepileptic drug. It is regarded as a first line drug in the treatment of patients suffering from partial seizures, with or without second generalizations, and in patients with generalized tonic-clonic seizures. Besides being an antiepileptic compound, carbamazepine has also proved to be effective in the treatment of trigeminal neuralgia and in patients suffering from manic-depressive illness, post therapeutic neuralgia, or phantom limb pain. The drug appears to act by reducing postsynaptic responses and by blocking post-tetanic potentiation. Chemically, it is 5H-dibenz[b,f]azepine-5-carboxamide and is disclosed in U.S. Pat. No. 2,948,718.

Although the half-life of carbamazepine is relatively long, ranging between 25 and 85 hours after a single dose, its effect is substantially reduced after repeated dosing due to autoinduction. Due to its increased metabolism, pronounced daily fluctuations in the serum concentration of carbamazepine are observed and are a cause for concern. The therapeutically effective blood levels of carbamazepine are from about 4 μg/ml to about 12 μg/ml. Blood levels of carbamazepine below 4 μg/ml are ineffective in treating clinical disorders, while levels above 12 μg/ml are most likely to result in side effects. The side effects are seen to a greater extent in syrup formulations due to the presence of fine particles of the active ingredient, which dissolve rapidly leading to faster drug absorption and higher peak plasma levels. The tablet formulations are relatively free of this disadvantage.

Modified release pharmaceutical compositions of carbamazepine produce substantial reduction in intra-dose fluctuations of carbamazepine concentrations, and thus tolerability. Hence seizure control in patients with epilepsy may be improved (Martindale—The Complete Drug Reference; 32^{nd} edition, ed. by Kathleen Parfitt, Pharmaceutical Press, 1999).

Extended release pharmaceutical compositions have been the focus of research for improved therapy, both through improved patient compliance resulting from the avoidance of missed doses through patient forgetfulness and decreased incidences of adverse drug reactions. It is the intent of extended release formulations to provide a longer period of pharmacological action after administration than what is ordinarily obtained after administration of immediate-release dosage forms.

Carbamazepine is commercially available in the US under the brand name Tegretol® as chewable tablets of 100 mg, immediate release tablets of 200 mg, extended release tablets of 100, 200, 400 mg and suspension of 100 mg/5 ml. Carbamazepine is also approved by the USFDA as extended release capsules of strengths of 100, 200, 300 mg and is sold under the brand names of Carbatrol® and Equetro®.

Tegretol® XR (extended-release tablets) contains a core and shell. The contents of the core are released through a small opening on one side. Fluid is absorbed through the shell, causing the contents to expand and slowly push out through the opening. Carbatrol® (extended-release capsule) is a multi-component formulation containing three different types of beads: immediate release, extended-release and enteric-release beads. The three different beads are combined in a specific ratio to provide twice-daily dosing.

Sustained release pharmaceutical compositions known in the art include specially coated pellets, coated tablets and capsules, and ion exchange resins, wherein the slow release of the active medicament is brought about through selective breakdown of the coating of the preparation or through formulating with a polymeric matrix to affect the release of a drug. Some sustained release formulations provide for pulsatile and/or sequential release of a single dose of an active compound at predetermined periods after administration.

For sustained-release pharmaceutical compositions containing very high quantities of the active pharmaceutical ingredient, it is particularly critical to avoid an excessively rapid release (dose dumping) as that can lead to undesirable toxic effects. Moreover, such systems are dependent upon gastric emptying rates and transit times, and can be associated with significant intra- and inter-individual variations.

These disadvantages have led to a shift in modified release technology from the use of monolithic systems to multiple unit systems in which each individual unit is formulated with modified release characteristics. The final dosage form includes a multiplicity of the individual units contained in a pharmaceutical composition in such a form that these individual units are made available from the formulation upon reaching the tract.

The expert is also aware that “multiple unit” pharmaceutical forms, in direct comparison with “single unit” pharmaceutical forms, are frequently evacuated substantially quicker from the stomach in the area of the upper small intestine (e.g. Bechgaard, Acta Pharmaceutica Technologica 28, No. 2, 1982, pages 149-157).

Multiple unit pharmaceutical compositions are usually contained within a rapid dissolving capsule, or are compressed into tablets. Soon after ingestion, upon dissolution of the capsule shell, the contents fall apart into the multiple units, such as beads or microtablets. Dispersion of drug over a large area in the gastrointestinal tract provides more time for the passage and aids in the absorption process. In addition, a multiple unit preparation is preferable to one single drug unit as the dose is spread out along the length of the intestine. In contrast to the single unit matrix tablets, each multiunit is coated with a polymeric controlled release film coating that provides the controlled, slowed release of active ingredient thereby all the sub-units in combination, result in desired slow release properties.

U.S. RE 34,990 discloses a core comprising carbamazepine and a protective colloid, a semi-permeable wall covering the drug-containing core and a passageway through the wall. Water permeates from the surrounding body fluids through the semi-permeable wall and the pressure that is built-up causes a suspension of the drug to be released from the passageway. A problem encountered with the osmotic system for carbamazepine was that when fine particles of anhydrous carbamazepine were used, upon contact with water, large needles of the dihydrate form of carbamazepine were formed. These crystals blocked the orifice of the osmotic system. Thus, osmotic system of US RE 34990 used hydroxypropyl methylcellulose as a protective colloid to prevent the conversion of the fine particles of anhydrous carbamazepine to the dihydrate form. However, even with the resolution of this problem the osmotic system has other disadvantages. Main concern is that the desired dose of carbamazepine should be pushed out of the osmotic system and become absorbed. The manufacture of oral osmotic drug delivery systems is complicated, involving procedures such as organic solvent based coating to form the semi-permeable membrane, and formation of the orifice or passageway using mechanical or laser drilling techniques. Particularly, the use of organic solvent-based coatings is undesirable due to environmental, safety and cost considerations.

U.S. Pat. No. 4,980,170 discloses a controlled release pharmaceutical composition of non-steroid anti-rheumatic, comprising a capsule, containing a combination of retarded release and gastric juice resistant pellets.

U.S. Pat. No. 5,326,570 discloses a drug delivery system comprising a combination of three types of units: an immediate release unit, a sustained release unit and an enteric release unit containing carbamazepine and said units are capable of releasing carbamazepine at varying times.

U.S. Pat. No. 5,912,013 discloses a pharmaceutical dosage form containing three different types of pellets enclosed in a hard gelatin capsule shell. These pellets essentially include atleast 70% carbamazepine as an active ingredient and 5% of polyvinyl pyrolidone as a binder. U.S. Pat. No. 5,955,104 discloses a multiple unit oral pharmaceutical dosage form consisting of pellets having a core and a coating wherein the core is inert and coating comprises drug layer, followed by second layer of release controlling polymer for retarding the release of the active ingredient.

U.S. Pat. No. 6,162,466 discloses a tablet for prolonged release of carbamazepine, comprising (a) a pharmaceutically effective amount of carbamazepine particles, (b) a methacrylic polymer and (c) additional excipients, wherein said methacrylic polymer and atleast one additional excipient form a single coating layer over said carbamazepine particles and atleast one additional excipient is present as an extragranular ingredient. The disclosure in the patent particularly points to the need to provide a single layer coating over the carbamazepine particles with methacrylic polymers. The said patent discloses only specific water insoluble grades of methacrylic acid polymer.

US 20070071819 discloses a multiple unit modified release carbamazepine compositions, including atleast one extended release unit and atleast one enteric release unit. The extended release core includes carbamazepine, one or more rate controlling polymers and one or more pharmaceutically acceptable excipients. The enteric release unit includes enteric polymer coating over a core comprising carbamazepine. The core of the enteric release unit may be an extended release core or an immediate release core.

Many of the available extended release pharmaceutical compositions of carbamazepine have inherent drawbacks of being expensive and require time-consuming methods of production. Accordingly, there is an unmet need in the art to develop additional extended release pharmaceutical compositions of carbamazepine which provide a therapeutically effective blood concentration level of carbamazepine for a sustained period and that involve simple and economical method of production.

### SUMMARY OF THE INVENTION

In accordance with a principal aspect of the present invention, there is provided an extended release pharmaceutical composition of carbamazepine or its pharmaceutical salts, solvates, or hydrates, wherein said composition is highly cost effective, as it comprises no sustained release polymeric coating. In addition, said composition provides better release profile employing less polymeric content.

In accordance with another aspect of the present invention, there is provided a simple and cost efficient process for manufacturing the extended release pharmaceutical composition of carbamazepine or its pharmaceutical salts, solvates, or hydrates.

In accordance with one preferred embodiment of the present invention, there is provided an extended release pharmaceutical composition comprising at least two populations of extended release minitablets each having a core comprising carbamazepine or its pharmaceutical salts, solvates, hydrates, and extended release polymer(s), wherein the extended release polymer of each population of minitablets is unalike.

In accordance with one preferred embodiment of the present invention, there is provided an extended release pharmaceutical composition comprising at least two populations of extended release minitablets, wherein the extended release polymer of at least one of the minitablet population is selected from group consisting of hydroxypropyl methylcellulose, methylcellulose, hydroxypropylcellulose, polyethylene oxide, cellulose acetate, ethyl cellulose, guar gum, locust bean gum, xanthan gum, karaya gum or any combination thereof and wherein the extended release polymer of at least another minitablet population is selected from group consisting of a methacrylate-based polymer, acrylate based polymer, acrylate/methacrylate based copolymers, and an ammonio acrylate/methacrylate copolymer or combination thereof.

In accordance with another preferred embodiment of the present invention, there is provided an extended release pharmaceutical composition comprising a first minitablet population and a second minitablet population, wherein each minitablet of the first and second minitablet population contains a core comprising carbamazepine or its pharmaceutical salts, solvates, or hydrates and extended release polymer(s) and wherein the extended release polymer of the first minitablet population and second minitablet population is unalike.

In accordance with another preferred embodiment of the present invention, there is provided an extended release pharmaceutical composition wherein the first minitablet population contains a core comprising carbamazepine or its pharmaceutical salts, solvates, or hydrates and ethylcellulose and second minitablet population contains a core comprising carbamazepine or its pharmaceutical salts, solvates, or hydrates and anionic copolymer based on methacrylic acid and ethyl acrylate.

In accordance with another preferred embodiment of the present invention, there is provided an extended release pharmaceutical composition, wherein first minitablet population contains a core comprising carbamazepine or its pharmaceutical salts, solvates, or hydrates, ethylcellulose and hydroxypropyl methylcellulose and second minitablet population contains a core comprising carbamazepine or its pharmaceutical salts, solvates, or hydrates, anionic copolymer based on methacrylic acid and ethyl acrylate and combination of ethylcellulose and hydroxypropyl methylcellulose.

In accordance with another preferred embodiment of the present invention, there is provided an extended release pharmaceutical composition, wherein the extended release composition is essentially a matrix based extended release composition.

In accordance with yet another preferred embodiment of the present invention, there is provided a process for manufacturing the extended release pharmaceutical composition of carbamazepine or its pharmaceutical salts, solvates, or hydrates, wherein the process comprises of preparing core minitablets of first and second minitablet population, optionally film coating the first and second minitablet population, and filling equal fraction of two minitablet population into suitable size capsules.

In accordance with another preferred embodiment of the present invention, there is provided an extended release pharmaceutical composition, wherein the minitablets population also comprises at least one or more additives and wherein the minitablets are filled into a capsule.

### BRIEF DESCRIPTION OF THE DRAWING

Further objects of the present invention together with additional features contributing thereto and advantages accruing there from will be apparent from the following description of preferred embodiments of the invention which are shown in the accompanying drawing FIGURES, wherein:

FIG. 1 is a graphical depiction of comparative biovailiabilty of commercially available carbamazapine extended release capsule “Carbatrol” from Shire and carbamazapine extended release capsule of example 1.

### DETAILED DESCRIPTION OF INVENTION

The present invention relates to an extended release pharmaceutical composition of carbamazepine or its pharmaceutical salts, solvates, or hydrates and process for manufacturing the same.

One embodiment of the present invention is an extended release pharmaceutical composition comprising at least two populations of extended release minitablets (first minitablet and second minitablet) each having a core comprising carbamazepine or its pharmaceutical salts, solvates, hydrates, an extended release polymer(s), and wherein the extended release polymer of each population of minitablets is unalike.

While the term “minitablet” is used to describe multi-particulate extended release pharmaceutical composition of the present invention, other multiparticulate forms of various sizes and shapes, including beads, pellets, spheroids, spheres, and granules are also contemplated as part of the present invention.

The term “extended release pharmaceutical composition” as used herein means a drug composition whose drug release characteristics with respect to time and for location are chosen so as to accomplish therapeutic or compliance objectives, not offered by conventional dosage forms. Such compositions provide significant advantages over conventional dosage forms in terms of reduced dosing frequency and mitigated side effects.

For the purpose of present invention, terms such as, “pharmaceutical composition”, “extended release pharmaceutical composition”, “pharmaceutical dosage form”, “composition” and “dosage forms”, have been used synonymously.

“Extended release polymer” as used herein means polymers which provide the extended release of the carbamazepine for period of at least 6 hours.

It must be noted that, as used in this specification and appended claims, the singular form ‘a’, ‘an’ and ‘the’ include plural referents unless the context clearly dictates otherwise. Thus for example, reference to an extended release polymer includes option of multiple polymers.

“Optional or optionally” as used herein means described circumstances may or may not occur, so that description include instances where the circumstances occurs and instances where it does not.

The term “about” as described in the specification and appended claims indicate that values slightly outside the cited values, i.e., plus or minus 0.1 to 10%, which are effective and safe.

Regardless of the type of minitablet population, the amount of carbamazepine or its pharmaceutical salts, solvates, and hydrates present in the core minitablets ranges from about 30% to about 90%, preferably from about 45% to about 75% by weight of the total pharmaceutical composition.

Minitablets of at least two population may contain one or more additives selected from the group consisting of extended release polymers, diluents, binders, surfactants, solubilizers, co-solvents, absorbents, colorants, dyes, permeation enhancers, stabilizers, osmotic agents, disintegrants, wetting agents, plasticizers, low viscosity polymeric film coating materials, tableting aids, glidants, lubricants, antistatic agents and dispersants or any combination thereof and aqueous or non-aqueous solvents for processing of the composition of the invention. Any additives used in the present invention must be pharmaceutically acceptable and compatible with carbamazepine or its pharmaceutical salts, solvates, or hydrates. Any combination of additives can be used for purpose of the present invention.

The amount of additives used in the minitablets of the present invention may range from about 10% to about 70% by weight based on the total weight of the composition.

First minitablet population comprises about 50% by weight of total minitablets filled in the capsule. First minitablet population comprises an amount of carbamazepine or its pharmaceutical salts, solvates, and hydrates from about 30% to about 90% by weight, preferably about 60 to 85%, and still more preferably about 70% to about 80%.

Extended release polymers of first minitablet population are selected from group consisting of hydroxypropyl methylcellulose, hydroxypropylcellulose, polyethylene oxide, cellulose acetate, ethyl cellulose, guar gum, locust bean gum, xanthan gum, karaya gum or any combination thereof. Preferably, amount of extended release polymer(s) for the first minitablet population; range from about 0.1% to about 20% by weight of first minitablet population, more preferably from about 1% to about 10%. Ethyl cellulose is the preferred choice of extended release polymer of the first minitablet population. Ethyl cellulose is commercially available under the brand name Ethocel® standard 45 premium from Dow Chemical Company, USA.

Second minitablet population comprises about 50% by weight of total minitablets filled in the capsule. Second minitablet population comprises an amount of carbamazepine or its pharmaceutical salts, solvates, and hydrates from about 30% to about 90% by weight, preferably about 60 to about 85%, and still more preferably about 70% to about 80%.

Preferably, pharmaceutical composition in the form of capsule of present invention comprises equal fraction of the first and second population of minitablets.

Extended release polymers of second minitablet population are selected from group consisting of a methacrylate-based polymer, acrylate based polymer, acrylate/methacrylate based copolymers and an ammonio acrylate/methacrylate copolymer or combination thereof.

The preferred polymer is anionic copolymer based on methacrylic acid and ethyl acrylate, and is commercially available as Eudragit® L100-55, from Degussa, Germany.

Preferably, amount of extended release polymer(s) ranges from about 0.1% to about 20% by weight of second minitablet population, more preferably from about 1% to about 10%.

Customarily, anionic copolymer based on methacrylic acid and ethyl acrylate are used as enteric, but inventors of present invention after considerable research have discovered that said polymers can function as extended release polymers when used uniquely in combination with other additives/excipients.

In accordance with another embodiment of the present invention, the extended release composition of carbamazepine or its pharmaceutical salts, solvates, or hydrates comprising two population of minitablets is essentially a matrix based extended release composition.

In accordance with another embodiment of the present invention, the extended release composition of carbamazepine or its pharmaceutical salts, solvates, or hydrates comprising at least two population of minitablets is essentially a matrix based extended release composition.

Preferably, extended release composition of the present invention comprises two minitablet population wherein first minitablet population contains a core comprising carbamazepine or its pharmaceutical salts, solvates, or hydrates and ethylcellulose and second minitablet population contains a core comprising carbamazepine or its pharmaceutical salts, solvates, or hydrates and anionic copolymer based on methacrylic acid and ethyl acrylate.

More preferably, extended release pharmaceutical composition comprises two minitablet population wherein first minitablet population contains a core comprising carbamazepine or its pharmaceutical salts, solvates, or hydrates, ethylcellulose and hydroxypropyl methylcellulose; second minitablet population contains a core comprising carbamazepine or its pharmaceutical salts, solvates, or hydrates, anionic copolymer based on methacrylic acid and ethyl acrylate and combination of ethylcellulose and hydroxypropyl methylcellulose.

Without being bound by any theory, extended release of the carbamazepine or its pharmaceutical salts, solvates, and hydrates from the core of minitablets of at least two populations is achieved from matrix formed of carbamazepine or its pharmaceutical salts, solvates, and hydrates with extended release polymers, and additives present in the core minitablets.

Matrix based extended release compositions are easier to make and offer significant advantages over reservoir type devices in terms of labour, equipment and efficiency.

Modulation of extended release of drug from the said minitablets is effected by type and concentration of the extended release polymer, in conjunction with the other pharmaceutical excipients present in the core minitablets.

Diluents used as part of the first the composition of the present invention may be any suitable diluent for use in pharmaceutical art, including but not limited to, lactose, microcrystalline cellulose, sucrose, dicalcium phosphate, fructose, mannitol, xylitol, sorbitol, starches and the like and mixtures thereof. Amount of diluents used ranges from about 5% to about 30% by total weight of the composition, preferably from about 10% to about 20%. The preferred diluents for the composition of the present invention are lactose or microcrystalline cellulose or combination thereof. Microcrystalline cellulose is commercially available under the brand name Avicel® from FMC Corporation, USA. Similarly, lactose is commercially available as Pharmatose 200 M from DMV-Fonterra, UK.

Binder used as part of the minitablets of the present invention may be any suitable binder for use in pharmaceutical art, including but not limited to, polyvinyl-pyrrolidine, hydroxypropyl methylcellulose, hydroxypropyl cellulose, methyl cellulose, sucrose, acacia, gelatin, pregelatinized starch, ethyl cellulose, sodium alginate or combinations thereof. The preferred binder for the composition of the present invention is low viscosity hydroxypropyl methylcellulose. Hydroxypropyl methylcellulose used as preferred binder for the present composition is commercially available under the brand name Pharmacoat® 606, from Aqualon, USA. Amount of binders used are in the range from about 1% to about 20% by total weight of the composition, preferably from about 2% to about 10%.

In one preferred embodiment, a combination of ethyl cellulose and hydroxypropyl methylcellulose is employed as binder for the second minitablet population of the extended release composition of the present invention. The preferred concentration of combination binder ranges from about 1% to about 5% by total weight of the minitablets of the second population. Ethyl cellulose for use as binder is commercially available from Dow Chemical Company, USA under the brand name Ethocel™ FP.

Surfactant used as part of the minitablets of the composition of the present invention may be any suitable surfactant for use in pharmaceutical art, including but not limited to, anionic surfactant, such as, sodium lauryl sulphate; cationic surfactant, such as, cetrimide and benzalkonium chloride; amphoteric surfactants, such as, N-Dodecyl N, N dimethyl betaine and Non-ionic surfactant such as, polyoxyethylated glycol monoethers, such as, cetomacrogols; sorbitan ethers and polysorbates or combinations thereof. Preferred surfactant is sodium lauryl sulphate. Surfactants may be present in the composition of the present invention in an amount of from about 0.1% to about 20% by total weight of the composition, preferably from about 0.5% to about 10%. Sodium lauryl sulphate is commercially available from Cognis Deutschland Gmbh, Germany under the trade name Texapon®K12 PPH.

Solubilizer used as part of the minitablets of the composition of the present invention may be any suitable solubilizer for use in pharmaceutical art include but not limited to, propylene glycol, polyethylene glycol, such as PEG-400, poloxamer, lecithin, polysorbate 80, and sodium lauryl sulphate or combinations thereof. Preferred solubilizer is polyethylene glycol (PEG-400). Solubilizers may be present in the composition of the present invention in an amount from about 0.1% to about 20% by total weight of the composition, preferably from about 0.5% to about 10%. Polyethylene glycol is commercially available as Lutrol® E 400 from BASF Pharma, Germany.

Optionally used stabilizers as part of the minitablets of the present invention may be any suitable stabilizer for use in pharmaceutical art, including but are not limited to, sodium sulfite, butylated hydroxy anisole, butylated hydroxy toluene, propyl gallate, sodium citrate, lactic acid and edetate disodium. Stabilizers may be present in the composition of the present invention in an amount of from about 0% to about 10% by total weight of the composition, preferably from about 0.5% to about 5%. Preferred stabilizer is sodium sulfite.

Glidants and antiadherants, which may be employed as part of the minitablets of the composition of the present invention, include, but are not limited to, talc, corn starch, silicon dioxide, and metallic stearates, such as magnesium and calcium stearates. Glidants may be used in the composition of the present invention in an amount of from about 0.2% to about 10% by total weight of the composition, preferably from about 0.5% to about 5%. Preferred glidant is talc.

Lubricants, which may be used in the composition of the present invention, include, but are not limited to, magnesium stearates, sodium stearates, calcium stearates, stearic acid, pregelatinised starch, sodium stearyl fumarate and, acrylate polymers, such as, Eudragit EPO or combination thereof. Lubricants may be present in the composition of the present invention in an amount of from about 0.2% to about 10% by total weight of the composition, preferably from about 0.5% to about 5%. Preferred lubricants are magnesium stearates and/or pregelatinised starch.

Minitablets of the present invention may also contain various pharmaceutically approved coloring and/or flavoring agents as known in the pharmaceutical art.

It may be desirable that minitablets of the two populations are film coated to improve its aesthetic appeal and the film coating in no way modulating the release of drug from the core of minitablets. The film coating material may be selected from group consisting of hydroxypropyl methylcellulose preferably low viscosity polymer, hydroxypropylcellulose and polyvinyl alcohol. The film coating material is dissolved in a suitable solvent (purified water), which may further contain suitable plasticizer, such as, propylene glycol, tributyl citrate, polyethylene glycol, castor oil, dibutyl sebacate, and glycerin; coloring agents such as, ferric oxide, red, yellow, or blends and antitacking agents such as talc, silicon dioxide and calcium silicate. Ready to use film coating material are available from Colorcon under the brand name Opadry™. Amount of film coating applied on core minitablets ranges from about 1% to about 5% by weight of the total minitablets.

In some embodiments, solvents used in processing of pharmaceutical composition of the present invention are purified water and isopropyl alcohol. Solvents used in the present invention are environment and pose no occupational hazards.

Finished extended released minitablets of at least two populations are filled in suitable size capsules in equal quantities. The minitablets are dispensed in the capsule of suitable size to make a suitable dosage form, wherein the dosage form provides a therapeutic level of carbamazepine or its pharmaceutical salts, solvates, and hydrates for at least six hours.

Extended release pharmaceutical composition of the present invention provides a dissolution profile in aqueous media such that about 25% to about 50% of carbamazepine is released after about 1 hour; about 55% to about 80% of carbamazepine is released after about 4 hours; and not less than 80% of carbamazepine is released after about 10 hours.

Dissolution studies of first and second minitablet population are also carried out. Dissolution profile of first minitablet population varies from the dissolution profile of second minitablet population.

In accordance with the present invention, a process for manufacturing extended release pharmaceutical composition of carbamazepine or its pharmaceutical salts, solvates, or hydrates comprises the steps of: preparing core minitablets of first and second minitablet population, optionally film coating the first and second minitablet population, and finally filing equal fraction of the two minitablet population into suitable size capsules and packing the said capsules in pharmaceutically acceptable packs.

The same manufacturing process can be employed for preparing the first and second population of minitablets of extended release composition of the present invention.

Minitablets of the composition of the present invention may be made by a direct compression, roll compaction or by a wet granulation method. Wet granulation is the preferred method for manufacturing the minitablets of at least two population of the composition of the present invention.

In wet granulation method, carbamazepine or its pharmaceutical salts, solvates, or hydrates after dry blending with other additives are granulated with a granulating liquid (e.g. isopropyl alcohol, and purified water) in a planetary mixer, high shear mixer or any fluidized bed processor. The binder may be present in the granulating agent or may be in the dry blend. The wet granules are dried in an oven or fluidized bed dryer, and then sieved through a suitable sieve to get free flowing minitablets or granules. The resulting minitablets or granules are blended with appropriate lubricant and glidants. Lubricated minitablets or granules so obtained are compressed into minitablets on a rotary press using appropriate tooling. If desired, a film coating can be applied onto compressed tablets. The compressed tablets from first and second population of minitablets are filled into capsules of suitable size.

Alternatively, after drying process, minitablets obtained from two different populations are dispensed into suitable size capsules.

The film coating of minitablets can be easily carried out in conventional perforated coating pans or in highly sophisticated fluid bed coater as is well known in the pharmaceutical art.

Studies, for evaluating the pharmacokinetic parameters of composition of the present invention vis-a-vis Carbatrol™ (US marketed extended release composition of carbamazepine from Shire Laboratories) was performed in 12 healthy volunteers. Various pharmacokinetic parameters evaluated are AUC, Cₘₐₓ and Tₘₐₓ. The area under the curve refers to area under the total carbamazepine plasma concentration time curve from time zero to the last quantifiable concentration. Similarly, Cₘₐₓ refers to peak plasma concentration level of carbamazepine on concentration time curve without interpolation. The ratio of area under the curve for the test product (Example 1 of the present invention) to reference product (Carbatrol™ 300 mg capsule) is determined to be within the range of 0.8 to 1.25. The ratio of peak plasma concentration of carbamazepine from test product (Example 1 of present invention) to reference product (Carbatrol™ 300 mg capsule) is also found to be within the range of 0.8 to 1.25. Compliance of the above two criteria testify that composition of the invention (two minitablet population) is bioequivalent to Carbatrol™. Being bioequivalent means that rate and extent of absorption of carbamazepine from two dosage forms are same.

The pharmaceutical composition of the at least dual minitablet population of the present invention eliminates the problem of dose dumping under fasting and fed conditions and further provides extended release of the drug from the dosage form in therapeutic range to enable twice a day dosage regimen of carbamazepine or its pharmaceutical acceptable salts. In addition, such a composition eliminates the side effects associated with the conventional instant release compositions of carbamazepine or its pharmaceutical salts, solvates, and hydrates.

The following examples further illustrate the invention and its unique characteristics in elaborate manner. However, following examples are not intended to limit the scope of invention in any way.

### Example 1

### (I) Composition of First Minitablet Population

| | | | | |
|---|---|---|---|---|
| | S. | | | |
| Stage | No. | Ingredients | Mg | % w/w |

Dry Blending

| | | | | |
|---|---|---|---|---|
| A | 1. | Carbamazepine | 300.00 | 73.53 |
| | 2. | Microcrystalline cellulose (Avicel-101) | 32.00 | 7.84 |
| | 3. | Ethyl Cellulose | 6.00 | 1.47 |
| | 4. | Lactose monohydrate | 24.50 | 6.00 |
| | | (Pharmatose 200m) | | |
| | 5. | Sodium lauryl sulphate (Texapon K12) | 5.50 | 1.35 |

Granulation

| | | | | |
|---|---|---|---|---|
| B | 1. | Hydroxypropylmethyl cellulose 6 cps | 20.00 | 4.90 |
| | | (Pharmacoat-606) | | |
| | 2. | Polyethylene glycol-400 | 2.00 | 0.49 |
| | 3. | Purified water | q.s | |

Lubrication

| | | | | |
|---|---|---|---|---|
| C | 1. | Pregelatinised starch (Lycatab) | 6.00 | 1.47 |
| | 2. | Talc | 6.00 | 1.47 |
| | 3. | Magnesium Stearate | 6.00 | 1.47 |

| | | |
|---|---|---|
| Total wt. | 408.00 | 100.0 |

### Manufacturing Procedure:

1. Carbamazepine, microcrystalline cellulose, lactose monohydrate, sodium lauryl sulphate and ethyl cellulose were passed through a suitable size mesh and transferred into RMG and blended together.
2. Hydroxypropyl methylcellulose and polyethylene glycol-400 were dissolved in purified water.
3. Blend obtained in step-1 was granulated with step-2 solution.
4. Wet mass obtained in step-3 was milled using a suitable size mesh
5. Wet granules obtained in step-4 were dried in rapid dryer.
6. Dried granules obtained in step-5 were milled using a suitable size mesh.
7. Pregelatinised starch, talc and magnesium stearate were passed through a suitable size mesh.
8. Dried granules obtained in step 6 were lubricated with pregelatinised starch, talc and magnesium stearate.
9. Lubricated granules obtained in step-8 were compressed into minitablets.

### (II) Composition of Second Population of Minitablet

| | | | | |
|---|---|---|---|---|
| | S. | | | |
| Stage | No. | Ingredients | Mg | % w/w |

Dry Blending

| | | | | |
|---|---|---|---|---|
| A | 1. | Carbamazepine | 300.00 | 73.53 |
| | 2. | Microcrystalline cellulose (Avicel-101) | 32.00 | 7.84 |
| | 3. | Lactose monohydrate | 24.30 | 5.96 |
| | | (Pharmatose 200m) | | |
| | 4. | Sodium lauryl sulphate (Texapon K120) | 5.50 | 1.35 |
| | 5. | Eudragit L-100-55 | 6.20 | 1.52 |

Granulation

| | | | | |
|---|---|---|---|---|
| B | 1. | Hydroxypropylmethyl cellulose 6 cps | 20.0 | 4.90 |
| | | (Pharmacoat-606) | | |
| | 2. | Ethyl cellulose | 6.00 | 1.47 |
| | 3. | Polyethylene glycol-400 | 2.00 | 0.49 |
| | 4. | Isopropyl alcohol | q.s | |
| | 3. | Purified water | q.s | |

Lubrication

| | | | | |
|---|---|---|---|---|
| C | 1. | Talc | 6.00 | 1.47 |
| | 2. | Magnesium stearate | 6.00 | 1.47 |

| | | |
|---|---|---|
| Total wt. | 408.00 | 100.0 |

### Manufacturing Procedure:

1. Carbamazepine, microcrystalline cellulose, lactose monohydrate, sodium lauryl sulphate and Eudragit L-100-55 were sifted through a suitable size mesh and transferred into RMG and blended together.
2. Hydroxypropylmethyl cellulose and polyethylene glycol-400 were dissolved in purified water.
3. Ethyl cellulose was dissolved in a mixture of isopropyl alcohol and purified water.
4. Blend obtained in step-1 was initially granulated with step-2 solution. The resultant granulate was again granulated with step-3 binder solution.
5. Wet mass obtained in step-4 was milled using a suitable size mesh.
6. Wet granules obtained in step-5 were dried in rapid dryer.
7. Dried granules obtained in step-6 were milled using a suitable size mesh.
8. Talc and magnesium stearate were sifted through a suitable size mesh and blended with dried granules obtained in step-7.
9. Lubricated granules obtained in step-8 were compressed into minitablets.

### (III) Formation of Carbamazepine ER Capsule

50% fraction of first population of minitablets as obtained above and 50% fraction of second population of minitablets as obtained above were blended to provide 300 mg dose of carbamazepine and filled into suitable size capsules.

### Example 2

### (I) Composition of First Minitablet Population

| | | | | |
|---|---|---|---|---|
| | S. | | | |
| Stage | No. | Ingredients | mg | % w/w |

Blending

| | | | | |
|---|---|---|---|---|
| A | 1. | Carbamazepine | 150.00 | 71.43 |
| | 2. | Microcrystalline cellulose (Avicel-101) | 16.00 | 7.62 |
| | 3. | Lactose monohydrate | 12.25 | 5.83 |
| | | (Pharmatose 200m) | | |
| | 4. | Hydroxypropylmethyl cellulose 6 cps | 10.00 | 4.76 |
| | | (Pharmacoat-606) | | |

Granulation

| | | | | |
|---|---|---|---|---|
| B | 1. | Sodium lauryl sulphate (Texapon K 12) | 2.75 | 1.31 |
| | 2. | Polyethylene glycol-400 | 1.00 | 0.47 |
| | 3. | Purified water | q.s | |
| C | 1. | Ethyl cellulose | 3.00 | 1.43 |
| | 2. | Isopropyl alcohol | q.s | |
| | 3. | Purified water | q.s | |

Lubrication

| | | | | |
|---|---|---|---|---|
| D | 1. | Pregelatinised starch (Lycatab) | 3.00 | 1.43 |
| | 2. | Talc | 3.00 | 1.43 |
| | 3. | Magnesium stearate | 3.00 | 1.43 |

Film coating

| | | | | |
|---|---|---|---|---|
| E | 1. | Opadry II white | 6.00 | 2.86 |
| | 2. | Purified water | q.s | |

| | | |
|---|---|---|
| Total wt. | 210.00 | 100.00 |

### Manufacturing Procedure:

1. Carbamazepine, microcrystalline cellulose, lactose monohydrate and hydroxypropylmethyl cellulose were passed through a suitable size mesh and transferred into RMG and blended together.
2. Sodium lauryl sulphate and polyethylene glycol-400 were dissolved in purified water.
3. Ethyl cellulose was dissolved in a mixture of isopropyl alcohol and purified water.
4. Premix obtained in step-1 was initially granulated with step-2 sodium lauryl sulphate solution, granulate so obtained was further granulated with step-3 binder solution.
5. Wet mass obtained in step-4 was milled using a suitable size mesh
6. Wet granules obtained in step-5 were dried in dryer.
7. Dried granules obtained in step-6 were milled using a suitable size mesh.
8. Pregelatinised starch, talc and magnesium stearate were passed through a suitable size mesh.
9. Dried granules obtained in step 7 were lubricated by blending with pregelatinised starch, talc and magnesium stearate.
10. Lubricated granules obtained in step-9 were compressed into minitablets.
11. Minitablets as obtained in step 10 were coated with 10% w/w solution of Opadry white in perforated coating pan.

### (II) Composition of Second Minitablets Population

| | | | | |
|---|---|---|---|---|
| | S. | | | |
| Stage | No. | Ingredients | mg | % w/w |

Dry blending

| | | | | |
|---|---|---|---|---|
| A | 1. | Carbamazepine | 150.00 | 71.43 |
| | 2. | Microcrystalline cellulose (Avicel-101) | 16.00 | 7.62 |
| | 3. | Lactose monohydrate | 12.15 | 5.79 |
| | | (Pharmatose 200m) | | |
| | 4. | Hydroxypropylmethyl cellulose 6 cps | 10.00 | 4.76 |
| | | (Pharmacoat-606) | | |
| | 5. | Eudragit L-100-55 | 3.10 | 1.48 |

Granulation

| | | | | |
|---|---|---|---|---|
| B | 1. | Sodium lauryl sulphate (Texapon K 12) | 2.75 | 1.31 |
| | 2. | Polyethylene glycol - 400 | 1.00 | 0.48 |
| | 3. | Purified water | q.s | |
| | 3. | Ethyl cellulose | 3.00 | 1.43 |
| | 4. | Isopropyl alcohol | q.s | |

Lubrication

| | | | | |
|---|---|---|---|---|
| C | 1. | Talc | 3.00 | 1.43 |
| | 2. | Magnesium stearate | 3.00 | 1.43 |

Film coating

| | | | | |
|---|---|---|---|---|
| D | 1. | Opadry II pink | 6.00 | 2.86 |
| | 2. | Purified water | q.s | |

| | | |
|---|---|---|
| Total wt. | 210.00 | 100.02 |

### Manufacturing Procedure:

1. Carbamazepine, microcrystalline cellulose, lactose monohydrate, hydroxypropylmethyl cellulose and Eudragit L-100-55 were sifted through a suitable size mesh and transferred into RMG and blended together.
2. Sodium lauryl sulphate and polyethylene glycol-400 were dissolved in purified water.
3. Ethyl cellulose was dissolved in a mixture of isopropyl alcohol and purified water.
4. Premix obtained in step-1 was initially granulated with step-2 sodium lauryl sulphate solution, granulate so formed was further granulated with step-3 binder solution.
5. Wet mass obtained in step-4 was milled using a suitable size mesh.
6. Wet granules obtained in step-5 were dried in dryer.
7. Dried granules obtained in step-6 were milled using a suitable size mesh.
8. Talc and magnesium stearate were sifted through a suitable size mesh and blended with dried granules obtained in step 7.
9. Lubricated granules obtained in step-8 were compressed into minitablets.
10. Minitablets as obtained in step 9 were coated with 10% w/w solution of Opadry pink in perforated coating pan.

### (III) Formation of Carbamazepine ER Capsule

50% fraction of first minitablet population as obtained above and 50% fraction of second minitablet population as obtained above were blended to provide 300 mg dose of carbamazepine and filled into suitable size capsules.

### Example 3

### (I) First Minitablet Population

| | | | | |
|---|---|---|---|---|
| | S. | | | |
| Stage | No. | Ingredients | Mg | % w/w |

Dry Blending

| | | | | |
|---|---|---|---|---|
| A | 1. | Carbamazepine | 300.00 | 73.26 |
| | 2. | Microcrystalline cellulose (Avicel-101) | 32.00 | 7.81 |
| | 3. | Lactose monohydrate | 27.00 | 6.59 |
| | | (Pharmatose 200 m) | | |
| | 4. | Hydroxypropyl methylcellulose 6 cps | 20.00 | 4.88 |
| | | (Pharmacoat-606) | | |

Granulation

| | | | | |
|---|---|---|---|---|
| B | 1. | Sodium lauryl sulphate (Texapon K 12) | 5.50 | 1.34 |
| | 2. | Polyethylene glycol-400 | 2.00 | 0.49 |
| | 3. | Purified water | q.s | |
| C | 1. | Ethyl cellulose 10 cps | 3.00 | 0.73 |
| | 2. | Isopropyl alcohol | q.s | |
| | 3. | Purified water | q.s | |

Lubrication

| | | | | |
|---|---|---|---|---|
| D | 1. | Eudragit EPO | 4.00 | 0.98 |
| | 2. | Pregelatinised starch (Lycatab) | 4.00 | 0.98 |
| | 3. | Magnesium stearate | 6.00 | 1.47 |
| | 4. | Talc | 6.00 | 1.47 |

| | | |
|---|---|---|
| Total wt. | 409.5 | 100.0 |

### Manufacturing Procedure:

1. Carbamazepine, microcrystalline cellulose, lactose monohydrate, and hydroxypropylmethyl cellulose were sifted through a suitable size mesh and transferred into RMG and blended together.
2. Sodium lauryl sulphate and polyethylene glycol-400 were dissolved in purified water.
3. Ethyl cellulose was dissolved in a mixture of isopropyl alcohol and purified water.
4. Premix obtained in step-1 was initially granulated with step-2 sodium lauryl sulphate solution, granulate so obtained was granulated with step-3 binder solution.
5. Wet mass obtained in step-4 was milled using a suitable size mesh.
6. Wet granules obtained in step-5 were dried in rapid dryer.
7. Dried granules obtained in step-6 were milled using a suitable size mesh.
8. Eudragit EPO, pregelatinised starch, magnesium stearate and talc were sifted through a suitable size mesh and blended with dried granules obtained in step 7.
9. Dried granules obtained in step-7 were blended with Eudragit EPO and Lycatab Pregelatinised starch.
10. Lubricated granules obtained in step-9 were compressed into minitablets.

### (II) Composition Second Minitablet Population

| | S. | | | |
|---|---|---|---|---|
| Stage | No. | Ingredients | mg | % w/w |
| A | 1. | Carbamazepine | 300.00 | 73.26 |
| | 2. | Microcrystalline cellulose (Avicel-101) | 32.00 | 7.81 |
| | 3. | Lactose monohydrate | 24.60 | 6.00 |
| | | (Pharmatose 200m) | | |
| | 4. | Hydroxypropylmethyl cellulose 6 cps | 20.00 | 4.88 |
| | | (Pharmacoat-606) | | |
| | 5. | Eudragit L-100-55 | 6.40 | 1.56 |

Granulation

| | | | | |
|---|---|---|---|---|
| B | 1. | Sodium lauryl sulphate (Texapon K 12) | 5.50 | 1.34 |
| | 2. | Polyethylene glycol-400 | 2.00 | 0.50 |
| | 3. | Purified water | q.s | |
| C | 1. | Ethyl cellulose | 3.00 | 0.73 |
| | 2. | Isopropyl alcohol | q.s | |
| | 3. | Purified water | q.s | |

Lubrication

| | | | | |
|---|---|---|---|---|
| D | 1. | Pregelatinised starch (Lycatab) | 4.00 | 0.98 |
| | 2. | Magnesium stearate | 6.00 | 1.47 |
| | 3. | Talc | 6.00 | 1.47 |

| | | |
|---|---|---|
| Total wt. | 409.5 | 100.0 |

### Manufacturing Procedure:

1. Carbamazepine, microcrystalline cellulose, lactose monohydrate, hydroxypropylmethyl cellulose and Eudragit L-100-55 were sifted through a suitable size mesh and transferred into RMG and blended together.
2. Sodium lauryl sulphate and polyethylene glycol-400 were dissolved in purified water.
3. Ethyl cellulose was dissolved in a mixture of isopropyl alcohol and purified water.
4. Premix obtained in step-1 was initially granulated with step-2 sodium lauryl sulphate solution. This was blended and was again granulated with step-3 binder solution.
5. Wet mass obtained in step-4 was milled using a suitable size mesh.
6. Wet granules obtained in step-5 were dried in rapid dryer.
7. Dried granules obtained in step-6 were milled using a suitable size mesh.
8. Pregelatinised starch, magnesium stearate and talc were sifted through a suitable size mesh.
9. Dried granules obtained in step 7 were blended with pregelatinised starch and finally lubricated with talc and magnesium stearate.
10. Lubricated granules obtained in step-9 were compressed into minitablets.

### (III) Formation of Carbamazepine ER Capsule

Procedure followed is same as described above in example 2.

### Example 4

### (I) Composition of First Minitablet Population

| | | | | |
|---|---|---|---|---|
| | S. | | | |
| Stage | No. | Ingredients | Mg | % w/w |

Blending

| | | | | |
|---|---|---|---|---|
| A | 1. | Carbamazepine | 300.00 | 73.53 |
| | 2. | Microcrystalline cellulose (Avicel-101) | 32.00 | 7.84 |
| | 3. | Lactose monohydrate | 28.50 | 6.94 |
| | | (Pharmatose 200m) | | |
| | 4. | Sodium lauryl sulphate (Texapon K12) | 5.50 | 1.35 |

Granulation

| | | | | |
|---|---|---|---|---|
| B | 1. | Hydroxypropylmethyl cellulose 6 cps | 20.00 | 4.90 |
| | | (Pharmacoat-606) | | |
| | 2. | Polyethylene glycol - 400 | 2.00 | 0.49 |
| | 3. | Purified water | q.s | |
| C | 1. | Ethyl cellulose | 6.00 | 1.47 |
| | 2. | Isopropyl alcohol | q.s | |
| | 3. | Purified water | q.s | |

Lubrication

| | | | | |
|---|---|---|---|---|
| D | 2. | Pregelatinised starch | 2.00 | 0.49 |
| | 3. | Talc | 6.00 | 1.47 |
| | 4. | Magnesium stearate | 6.00 | 1.47 |

| | | |
|---|---|---|
| Total wt. | 408.0 | 100.0 |

### Manufacturing Procedure:

Same as described in example 3 above.

### (II) Composition of Second Minitablet Population

| | | | | |
|---|---|---|---|---|
| | S. | | | |
| Stage | No. | Ingredients | Mg | % w/w |

Dry Blending

| | | | | |
|---|---|---|---|---|
| A | 1. | Carbamazepine | 300.00 | 73.53 |
| | 2. | Microcrystalline cellulose (Avicel-101) | 32.00 | 7.84 |
| | 3. | Lactose monohydrate | 24.30 | 5.96 |
| | | (Pharmatose 200m) | | |
| | 4. | Hydroxypropylmethyl cellulose 6 cps | 20.00 | 4.90 |
| | | (Pharmacoat-606) | | |
| | 5. | Eudragit L-100-55 | 6.20 | 1.52 |

Granulation

| | | | | |
|---|---|---|---|---|
| B | 1. | Sodium lauryl sulphate (Texapon K 12) | 5.50 | 1.35 |
| | 2. | Polyethylene glycol - 400 | 2.00 | 0.49 |
| | 3. | Ethyl cellulose | 6.00 | 1.47 |
| | 4. | Isopropyl alcohol | Q.s | |
| | 5. | Purified water | Q.s | |

Lubrication

| | | | | |
|---|---|---|---|---|
| D | 1. | Talc | 6.00 | 1.47 |
| | 2. | Magnesium stearate | 6.00 | 1.47 |

| | | |
|---|---|---|
| Total wt. | 408.0 | 100.0 |

### Manufacturing Procedure:

Same as described in example 3 above.

### (III) Procedure for Formation of Carbamazepine ER Capsule

Same as described in example 3 above.

### Example 5

To assess the release of drug substance (Carbamazepine) from the first and second minitablet population, individual units of extended release carbamazepine capsule of Example 1 were subjected to in vitro dissolution testing. The results obtained from dissolution profile of each individual unit, i.e. first and second minitablet population were presented in table as a mean percentage release of the total carbamazepine. Dissolution study parameters were as follows:

Instrument parameters: USP type II; 75 rpm
Dissolution parameters: First 4 hrs: 600 ml of dilute acid (pH 1.1) with 1.8% beta-cyclodextrin.
After 4 hrs: 1000 ml of 50 mM phosphate buffer (pH 7.5) with 1.1% beta-cyclodextrin

TABLE 2
Dissolution profile

Mean percentage release of drug (carbamazepine)

| | First minitablet | Second minitablet |
|---|---|---|
| Time (hr) | population of Example 1 | population of Example 1 |
| 0 | 0 | 0 |
| 1 | 29 | 8 |
| 2 | 38 | 11 |
| 4 | 45 | 18 |
| 6 | 48 | 42 |
| 8 | 49 | 47 |
| 10 | 49 | 48 |

From the above tabular data, it is clearly evident that the dissolution profile of first minitablet population varies from the dissolution profile from second minitablet population.

To assess the release of drug substance (Carbamazepine) from the extended release pharmaceutical composition, extended release carbamazepine capsule of Example 1 was subjected to in vitro dissolution testing. The dissolution profile from extended release carbamazepine capsule of Example 1 was compared with the dissolution profile from the commercially available carbamazepine extended release capsule (Carbatrol® 300 mg from, USA.) The results are presented in table as a mean percentage release of the total carbamazepine from the extended release capsule. Dissolution study parameters were as follows:

Instrument parameters: USP type II; 75 rpm

Dissolution parameters: First 4 hrs: 600 ml of dilute acid (pH 1.1) with 1.8% beta-cyclodextrin.

After 4 hrs: 1000 ml of 50 mM phosphate buffer (pH 7.5) with 1.1% beta-cyclodextrin.

TABLE 1
Dissolution profile

Mean percentage release of drug (carbamazepine)

| | | Carbamazepine extended release |
|---|---|---|
| Time (hr) | Carbatrol ® ER | capsule of Example 1 |
| 0 | 0 | 0 |
| 1 | 39 | 35 |
| 2 | 58 | 54 |
| 4 | 69 | 69 |
| 6 | 104 | 101 |
| 8 | 106 | 101 |
| 10 | 106 | 102 |

From the above tabular data, it is clearly evident that the carbamazepine extended release capsule of the invention (Ex. 1) have substantially the same dissolution profile as that of Carbatrol®ER.

### Example 6

The bioavailability of the oral extended release pharmaceutical composition of carbamazepine and that of marketed extended release composition of carbamazepine were studied. A single dose, open label, randomized, two-way crossover study was carried out under fed condition for the same for the same. Carbatrol® 300 mg extended release capsules were used as the reference standard.

The pharmacokinetic assessment was based on the plasma level of carbamazepine measured by blood sampling. Blood samples were obtained before dosing and at the following time points after administration of both the reference and test medications—2, 4, 6, 8, 10, 11, 12, 13, 14, 15, 16, 17, 18, 20, 24, 28, 32, 36, 48, 72, and 120 hours post dose.

Twelve healthy volunteers were enrolled for the study but 10 were able to complete both the periods. After an overnight fast for at least 10 hours, subjects were served USFDA recommended (with respect to calorie content) high fat vegetarian breakfast, which they were required to consume completely within 30 minutes of serving the same. Subjects were administered a single extended release capsule of carbamazepine (300 mg, Example 1) in sitting posture with 240 ml drinking water at 30 minutes after serving of the USFDA recommended (with respect to calorie content) high fat vegetarian breakfast at ambient temperature, as the test medication while a single capsule of Carbatrol® 300 mg was administered as reference medication. Drinking water was prohibited 1 hr before dosing and 2 hrs after dosing. Standard meals were provided at appropriate time during both the periods.

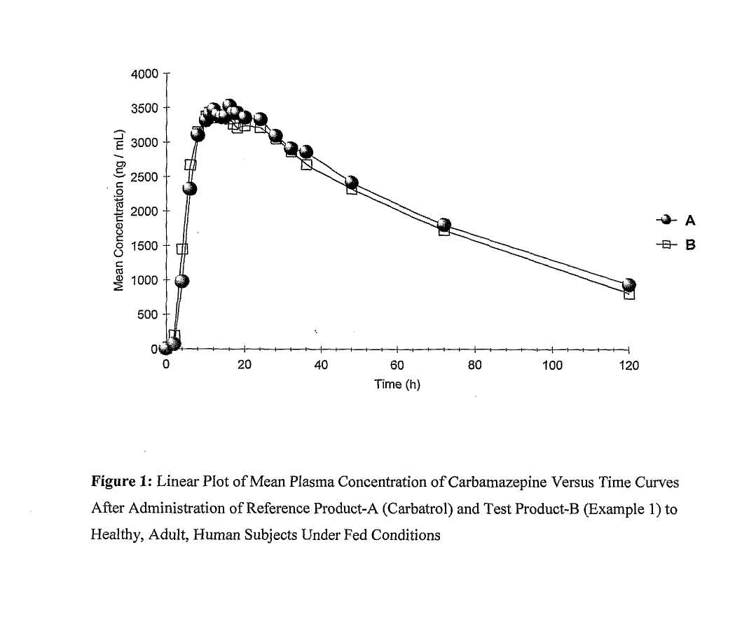

The plasma concentration of carbamazepine was determined at different time points and averaged over 10 volunteers. The plasma concentration profiles have been depicted in FIG. 1. Pharmacokinetic parameters for both reference and test formulations Cₘₐₓ, AUC₀₋ₜ, AUC_{0-∞} for the test and reference have been shown in Table 3

TABLE 3

| | Reference Product | Test Example | 90% Confidence Interval | |
|---|---|---|---|---|
| Parameters | Carbatrol-R (Mean) | 1-T (Mean) | (Test vs Reference) | Power |
| Cₘₐₓ (ng/ml) | 3778.9 | 3569.2 | 87.77-103.74 | 99.5 |
| AUC₀₋ₜ (ngh/ml) | 248766 | 240345 | 90.19-103.67 | 99.8 |
| AUC_{0-∞} (ngh/ml) | 326073 | 299042 | 86.46-99.58 | 99.8 |

Based on the above results extended release pharmaceutical composition of the present invention was found to be bioequivalent to Carbatrol® 300 mg.

### INDUSTRIAL APPLICATION

The extended release compositions of carbamazepine or its pharmaceutically acceptable salts of present invention are orally useful for the treatment of epilepsy, including grand mal epilepsy. Solid oral compositions of present invention are also useful for the treatment of pain associated with true trigeminal neuralgia and glossopharyngeal neuralgia. These solid oral compositions are provided in 100, 200 and 300 mg strengths.

Although the invention herein has been explained with reference to particular embodiments and examples, it is to be appreciated that these embodiments and examples are simply illustrative of principles and applications of the present invention. It is therefore to be understood that numerous modifications may be carried out to the illustrative embodiments and examples without departing from the spirit and scope of the present invention as defined by appended claims.

We claim:

1. An extended release pharmaceutical composition comprising at least two populations of extended release minitablets each having a core comprising carbamazepine or its pharmaceutical salts, solvates, hydrates, an extended release polymer(s), wherein the extended release polymer of each population of minitablets is unalike.

2. The extended release pharmaceutical composition of claim 1, wherein the extended release polymer of at least one of the minitablet population is selected from group consisting of hydroxypropyl methylcellulose, methylcellulose, hydroxypropylcellulose, polyethylene oxide, cellulose acetate, ethyl cellulose, guar gum, locust bean gum, xanthan gum, karaya gum or any combination thereof and wherein the extended release polymer of at least another minitablet population is selected from group consisting of a methacrylate-based polymer, acrylate based polymer, acrylate/methacrylate based copolymers, and an ammonio acrylate/methacrylate copolymer or combination thereof.

3. The extended release pharmaceutical composition of claim 2, wherein the extended release polymer of at least one of the minitablet population is ethyl cellulose.

4. The extended release pharmaceutical composition of claim 2, wherein the extended release polymer of at least another minitablet population is anionic copolymer based on methacrylic acid and ethyl acrylate.

5. The extended release pharmaceutical composition of claim 1, wherein the composition comprising first minitablet population and second minitablet population each having a core comprising carbamazepine or its pharmaceutical salts, solvates, hydrates, an extended release polymer(s), wherein the extended release polymer of each population of minitablets is unalike.

6. The extended release pharmaceutical composition of claim 5, wherein the extended release polymer of first minitablet population is selected from group consisting of hydroxypropyl methylcellulose, methylcellulose, hydroxypropylcellulose, polyethylene oxide, cellulose acetate, ethyl cellulose, guar gum, locust bean gum, xanthan gum, karaya gum or any combination thereof.

7. The extended release pharmaceutical composition of claim 6, wherein the extended release polymer of first minitablet population is ethyl cellulose.

8. The extended release pharmaceutical composition of claim 5, wherein the extended release polymer of second minitablet population is selected from group consisting of a methacrylate-based polymer, acrylate based polymer, acrylate/methacrylate based copolymers, and an ammonio acrylate/methacrylate copolymer or combination thereof.

9. The extended release pharmaceutical composition of claim 8, wherein the extended release polymer of second minitablet population is anionic copolymer based on methacrylic acid and ethyl acrylate.

10. The extended release pharmaceutical composition of claim 1, wherein the minitablets are filled into capsules.

11. The extended release pharmaceutical composition of claim 1, wherein the extended release minitablets further comprises additives selected from the group consisting of diluents, binders, surfactants, solubilizer, co-solvents, absorbents, colorants, dyes, permeation enhancers, stabilizers, osmotic agents, disintegrants, wetting agents, plasticizers, low viscosity polymeric film coating materials, tableting aids, glidants, lubricants, plasticizers, antistatic agents, dispersants or any combination thereof.

12. The extended release pharmaceutical composition of claim 1, wherein the composition is a matrix based extended release composition

13. A process for manufacturing extended release pharmaceutical composition of claim 1, the process comprising the steps of preparing core minitablet of at least two minitablets population, optionally film coating the same and filling equal fraction of the minitablet population into suitable size capsules.
